(19) United States
(12) Patent Application Publication
(10) Pub. No.: US 2004/0167162 A1
(43) Pub. Date: Aug. 26, 2004

(54) Uses for anti-malarial therapeutic agents

(76) Inventor: B. .Lauren Charous, Fox Point, WI (US)

Correspondence Address: SCULLY SCOTT MURPHY & PRESSER, PC, 400 GARDEN CITY PLAZA, GARDEN CITY, NY, 11530

(21) Appl. No.: 10/293,770

(22) Filed: Nov. 12, 2002

Related U.S. Application Data
(60) Provisional application No. 60/345,872, filed on Nov. 9, 2001.

(51) Int. Cl. A61K 31/47

(52) U.S. Cl. 514/312; 514/313

(57) ABSTRACT

The present invention is directed to the use of anti-malarial compound for the treatment and prophylaxis of infections by adenovirus or rhinovirus.

### RELATED APPLICATIONS

The present application is claiming priority of provisional application U.S. S. No. 60/345,872, filed on Nov. 9, 2001.

### FIELD OF THE INVENTION

The present invention relates to a method for treating viral respiratory infections in a mammal suffering therefrom, which comprises administering by targeted organ delivery to said mammal an anti-viral effective amount of an anti-malarial compound such as hydroxychloroquine (HCQ). It is also directed to the use of the anti-malarial compounds for the treatment of colds.

### BACKGROUND OF THE INVENTION

“Most infections of the nasopharynx are caused by viruses and give rise to the signs and symptoms that are collectively known as the common cold. Approximately 40-50% of colds are caused by the rhinovirus (RV) group.” (Schaechter M, Engeleberg N, Eisenstein B, Medoff G, Mechanismsof Microbial Disease (3rd Edition). Lippincott, Williams, and Williams. Philadelphia, 1999, p550). Other important viral pathogens include human corona virus, adenoviruses and influenza viruses. Recent evidence also implicates rhinovirus infections as an important precipitating factor for exacerbations of asthma, chronic bronchitis, sinusitis and otitis media. To be considered as effective for treatment and prevention of acute respiratory illness, an anti-viral therapy must include demonstrated activity against RV.

Viral infections account for nearly 85% of acute asthmatic episodes in children and in approximately half of exacerbations in adult asthmatic subjects. RV is the most commonly detected virus (Grunberg, K et al., “Experimental rhinovirus 16 infections causes variable airway obstruction in subjects with atopic asthma”, Am J Respir Crit Care Med 1999;160:1375-80; Gem, J E et al., “The role of viral infections in the natural history of asthma”, J Allerg Clin Immunol, 2000; 106:201-12). The fluctuations of hospitalization rates of patients with chronic obstructive pulmonary disease correlate strongly with seasonal variations in viral infection rates (Johnston SL et al. Am J Respir Crit Care Med. 1996; 154:654). RV infections in elderly persons (ages range from 60-90 years) cause illness severity comparable to influenza with a mean duration of viral respiratory tract symptoms of 16 days and daily activity restrictions in more than a quarter of affected individuals (Nicholson K G et al. BMJ. 1996; 313:1119). Rhinoviruses are picomaviruses which are among the smallest RNA containing animal viruses. They have a particle mass of about 8.3×10⁶D, 30% of which is a single stranded RNA consisting of approximately 7500 nucleotides. They have an icoschedral protein shell, which is about 300 Angstroms in diameter, and which contains 60 protomers, each consisting of 4 structural proteins VP 1, VP2, VP3 and VP4. The arrangement is that there are 60 trimers of pseudo-equivalent VP 1, VP2 and VP3 subunits on the icoscahedral capsid. In this arrangement the 180 chemically identical subunits are quasi-symmetrically related to form a T=3 icosadeltahedron. These 4 proteins are synthesized by an infected cell as a single polypeptide, which is cleaved into the individual subunits during virion assembly.

The protein capsids of the rhinovirus form a hollow shell enclosing a disordered core made up of the neural RNA. The VP4 largely lines the inside of the capsid.

Epithelial cells which line the nasopharynx and bronchial airways are the primary site of rhinovirus infections. While the initial site of infection is most commonly the nasopharynx, recent research demonstrates that in experiment in in vivo RV infection, the lower respiratory bronchial epithelium is commonly involved as well. (Mosser, A G et al., “Similar frequency of rhinovirus-infectible cells in upper and lower airway epithelium”, J Infect Dis 2002; 185: 734-43). Entry into the epithelial cells occurs via the intracellular adhesion molecule-I (ICAM-1) which acts as the receptor for 90% of rhinoviruses. Infection of the cells induces up-regulation of the ICAM-1 receptor. (Grunberg K et al., “Experimental rhinovirus 16 infection increases ICAM-1 expression in bronchial epithelium of asthmatics regardless of inhaled steroid treatment”, Clin Exp Allergy 2000; 30:1015-23). RV requires an acid environment to enter the cytoplasm via endosomal vesicles.

In contrast to other respiratory viruses, such as influenza, cytotoxic damage of infected epithelial cells does not appear to play a role in the pathogenesis of the symptoms induced by rhinovirus infections, since cytotoxicity is not observed in either infected human epithelial cell cultures or in the nasal mucosa of infected individuals. Rather, symptoms are thought to result from the elaboration of pro-inflammatory mediators generated initially by epithelial cells. Later, these are augmented by the recruitment of leukocytes and other cells capable of generating an inflammatory response. Support for this hypothesis has come from two lines of evidence:

1) Studies of subjects with experimentally induced or naturally acquired colds have demonstrated increased levels of several cytokines, including kinins, IL-1, IL-6, TNF-α, G-CSF and INF-γ in nasal secretions during symptomatic rhinovirus infections which are released by the epithelial cells. The severity of respiratory symptoms correlates closely with increases in these cytokines. 2) Infection of purified human respiratory epithelial cell populations with rhinovirus has been shown to induce production of a group of potent pro-inflammatory CXC chemokines, such as IP-10 and RANTES, that play a central rote in initiating leukocyte recruitment.

Once recruited during the acute stages of the viral infection, neutrophils, eosinophils and mononuclear cells all participate in the inflammatory cascade by releasing superoxide, proteases and eosinophil granular proteins.

To date, however, the specific biochemical events involved in the production of each of these cytokines by rhinovirus-infected epithelial cells are incompletely understood and the role of specific cytokines and other mediators, in the pathogenesis of cold symptoms remains to be established. The active role of leukocytes in the inflammatory cascade moreover, suggests that an effective drug must in some way blunt the contribution of these inflammatory cells as well. As a result, it has been difficult to find a therapeutic agent which is effective for treating and/or preventing the diseases caused by or associated with rhinovirus infections. A further obstacle to treatment is the rapid onset of infection; symptoms typically appear within twenty-four to forty-eight hours after viral innoculation. To be successful, acute treatment given to prevent symptoms must reach therapeutic concentrations within this interval. Alternatively, treatment must be of sufficient safety to be administered chronically prior to infection.

Adenoviruses are also common human pathogens. They are a major cause of respiratory and gastrointestinal infections as well as infections of the heart. Adenoviruses are widespread in nature.

Adenoviruses are simple non-enveloped DNA-containing viruses (i.e., composed of only DNA and protein) that multiply in the cell nucleus of the host.

The viral particles of the adenovirus have a dense central core and an outer coat known as the capsid. These particles have an icosahedral configuration and are composed of 252 capsomers, 240 hexons make up the faces and edges of the equilateral triangles and 12 pentons comprise the vertices. The hexons are truncated triangular or polygonal prisms with a central hole. The pentons are more complex consisting of a polygonal base with an attached fiber protein, whose length (i.e., short or long) varies with viral type. Minor capsid proteins are also associated with the hexons or pentons and confer stability on the capsid to form links with the core proteins and to function in virion assembly.

Each virion contains one linear, double-standard DNA molecule associated with proteins to form the core of the adenovirus.

They are a major cause of respiratory and gastrointestinal infections as well as infections of the heart. These viruses induce latent or acute infections in tonsils, adenoids, lungs, bladder and cornea as well as the gastrointestinal tract and are readily activated. Moreover, a type of adenoviruses, the enteric adenoviruses, such as Adenovirus Type 40 or 41 (and also known as Type F Enteric Adenovirus) are a virus group that causes serious intestinal and diarrheal diseases of young children.

However, the control of adenoviruses, e.g., enteric adenoviruses which are responsible for at least 15% of all cases of severe infantile gastroenteritis, is not within reach. In addition, adenoviruses are responsible for 5% of the acute respiratory infections in children under 4 years of age and are found in 10% of the respiratory diseases in this age group requiring hospitalization. Such conditions are generally associated with pharyngitis, coughing and conjunctivitis. Very often laryngotracheobronchitis occurs which develops into pneumonia in young children, and in fact 10% of childhood pneumonia are due to adenovirus infections and are often fatal in children under 2 years of age.

In the older population, adenoviruses are often responsible for pharyngoconjunctival fever and acute respiratory disease in institutionalized persons where it has been known to have a fatal outcome. The viruses are often associated with pertussis syndrome, haemorrhagic cystitis, meningitis, diarrhea and epidemic kerato conjunctivitis. The latter condition is characterized by rapid conjunctival involvement with pain, photophobia, lymphadenopathy and subsequent keratitis. The patient is therefore disabled to varying degrees over a period of time. It has been shown that adenovirus type 8 is the major etiologic agent in this particular aspect of adenovirus disease. Adenovirus disease is particularly severe in children with severe combined immunodeficiency disease (SCID) and in immunocompromised hosts. Adenoviruses are recognized also as increasingly more common in patients with Acquired Immune Deficiency Syndrome (AIDS) and in bone marrow transplant recipients.

The mechanism by which viruses infect mammalian cells is not uniform. Many viruses require a low pH in endosomal vesicles during cellular entry and incorporation. Adenovirus (Bartlett J S et al., “Infectious entry pathway of aeno-associated virus and adeno-associated virus vectors”, J Virol 2000; 74: 2777-85; Tibbles L A et al., “Activation of p38 and ERK signaling during adenovirus vector cell entry lead to expression of the CXC chemokine IP-10”, J Virol, 2002; 76: 1559-68), human corona virus (Hansen G H et al., “The cornavirus transmissible gastroenteritis virus causes infection after receptor-mediated endocytosis and acid-dependent fusion with an intracellular compartment”, J Virol, 1998; 72: 527-34) and influenza virus (Guinea R et al., “Requirement for vacuolar proton-ATPase activity during entry of influenze virus into cells”, J Virol 1995; 69: 2306-12) all require acidified endosomes for viral entry. Substances which elevate endosomal pH profoundly inhibit viral infectivity. Bafilomycin A1 (Perez L et al. “Entry of poliovirus into cells does not require a low-pH step”, J Virol 1993; 67: 4543-8), ammonium chloride (Li D et al., “Role of pH in syncytium induction and genome uncoating of avian infectious bronchitis corona virus (IBV)”, Adv Exp Med Biol 1990; 276: 33-6), and the anti-malarial agents chloroquine and hydroxychloroquine (Sperber K et al., “Inhibition of human immunodeficiency virus type 1 replication by hydroxychloroquine in T cells and monocytes”, AIDS Res Hum Retroviruses 1993; 9: 91-8) all exert their anti-viral effects by inhibiting endosomal acidification.

Treatment with bafilomycin A1, ammonium chloride or chloroquine block cellular infection by adenovirus (Bartlett J S op cit., Tibbles L A, op cit., Sperber op cit., Zsengeller Z et al., “Internalization of adenovirus by alveolar macrophages initiates early pro-inflammatory signaling during acute respiratory tract infection”, J Virol 2000; 74: 9655-67); and by influenza viruses (Ochiai H, “Inhibitory effect of bafilomycin A1, a specific inhibitor of vaculolar-type proton pump, on the growth of influenza A and B viruses in MDCK cells”, Antiviral Res 1995; 27: 425-30, Guinea, R op cit., Shibata M, “Mechanism of uncoating of influenza B virus in MDCK cells: action of chloroquine”, J Gen Virol 1983; 64: 1149-56). Utilizing the same mechanism of action, bafilomycin A1 and ammonium chloride block infection of human corona virus (Hansen, G H op cit.).

In addition, bafilomycin A1 has been shown to inhibit infection by rhinovirus (Perez L et al., “Entry of poliovirus into cells does not require a low-pH step”, J Virol 1993; 67: 4543-8) and, in particular, of human tracheal epithelial cells by the rhinovirus group 14 (Suzuki T et al. “Bafilomycin A(1) inhibits rhinovirus infection in human airway epithelium: effets on endosome and ICAM-1”, Am J Physiol Lung Cell Mol Physiol 2001; 280: L1115-27). Bafilomycin A1 also reduces expression of ICAM-1, the rhinovirus receptor in treated cells.

Nevertheless, there has hitherto been no useful antiviral compound that is effective in the treatment and/or prophylaxis of viral infections in the clinical setting, including rhinovirus or adenovirus infections, and there is also no adequate vaccine.

Thus, there is a need to find a drug which is useful for treating diseases or maladies caused by or associated with an infection by a virus, such as adenovirus or a rhinovirus. In addition, there is also a need to find a drug which is useful for the prophylaxis of diseases or maladies caused by or associated with an infection by a virus, e.g., such an adenovirus or rhinovirus.

The present inventor has found compounds which are useful for both the prophylaxis and treatment of maladies or diseases caused by or associated with these viral infections. He has found that compounds which have been known to be useful for treating malaria and which also have other anti-inflammatory activities are effective in treating and/or preventing these viral infections and the diseases caused by or associated with these viral infections. In addition, the present inventor describes a method of drug administration of these compounds which facilitates rapid onset of action of these agents.

### SUMMARY OF THE INVENTION

It is therefore an object of the present invention to treat or prevent common colds in a mammal, including humans, by administering thereto anti-malarial compounds in amounts effective to treat the cold or in prophylactically effective amounts, respectively.

The present invention is also directed to the treatment of viral infections in a mammal which comprises administering by targeted organ delivery to said mammal an anti-viral effective amount of an anti-malarial compound. By administering these compounds by targeted organ delivery, such as by inhalation, powders or mists or by use of a nasal spray containing these anti-malarials, a novel method for administering therapeutic drug concentrations are achieved in the nasopharygeal and bronchial airway linings within a timeframe suitable to ablate or minimize symptoms due to viral infection. This method has the advantage not only of providing rapid oset of action as compared to oral dosing but also of decreasing dosage requirements to less than twenty percent of conventional oral dosing. In another embodiment, the present invention is directed to the prophylaxis of viral infections in a mammal which comprises administering by targeted organ delivery to said mammal a prophylactically effective amount of an anti-malarial compound.

### BRIEF DESCRIPTION OF THE DRAWINGS

FIG. 1 graphically depicts the effect of 50 μm HCQ in the elaboration of IP-10 and RANTES in primary human epithelial cells with exposure to human rhinovirus type 16.

FIG. 2 depicts graphically the effect of varying concentrations of HCQ preincubation on the elaboration of EP-1 and RANTES in BEAS-2B epithelial cells exposed to human rhinovirus-16.

FIG. 3 graphically depicts the effect of HCQ on Eosinophil total Superoxide production. In FIG. 3, nil refers to control, i.e., the absence of HCQ; PMA refers to phorbol myristic acetate; PAF refers to platelet activating factor, and SE refers to standard error of the mean. The data in FIG. 3 are presented as means±SE; n=3. The * indicates p<0.05; ** indicates p<0.01.

FIG. 4 graphically depicts the mean whole blood concentration of HCQ following single day intravenous doses to male and female rats.

FIG. 5 graphically depicts the mean whole blood concentration of HCQ following single day intravenous doses to male and female dogs.

### DETAILED DESCRIPTION OF THE INVENTION

The present inventor has discovered that anti-malarial compounds including most specifically those of the quinoline class, possess the ability to:

1] inhibit viral infection of airway epithelial cells by rhinovirus, adenovirus, human corona virus, and influenza by altering the pH of entry vesicles and by decreasing expression of ICAM-1 which is the rhinovirus cell surface receptor;

2] abrogate the elaboration of symptom-inducing pro-inflammatory chemokines from epithelial cells infected with rhinovirus and;

3] inhibit the production of inflammatory mediators from leukocytes recruited to the site of viral inflammation.

The present inventor has found that the anti-malarial compounds are most effective in treating viral infections or preventing viral infections when administered by targeted organ delivery. As used herein, the term “targeted organ delivery” refers to the direct administration to the organ which is infected by the viruses. Viral infections, resulting in colds or other respiratory tract infections usually infect the pulmonary system, including by not limited, to the nose, throat, lungs, and the like. In addition, they also may infect the eyes and ears. When administered in anti-viral effective amounts by targeted organ delivery, the anti-malarial compounds are useful in treating viral infections. Moreover, the anti-malarial compounds when administered by direct targeted organ delivery are useful in preventing viral infections when administered in prophylactically effective amounts.

The present inventors have found that the direct targeted delivery of the anti-malarial compounds is the most effective way of administering these drugs. For example, as described hereinbelow, when administered directly by targeted organ delivery, the concentration of the anti-malarial compounds at the infected organs is maximized. When they are administered by other means as, for example, orally the concentration of the anti-malarial compounds that reaches the infected organ is significantly less. Consequently, when administered by targeted organ delivery, the anti-malarial compounds are more effective. Moreover, significantly less anti-malarial compounds are required for efficacious results when administered by targeted organ delivery than by oral administration.

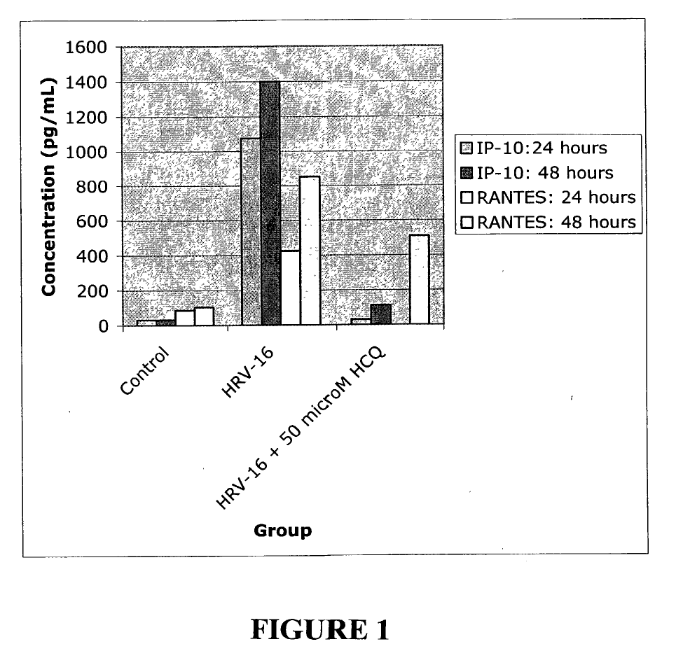

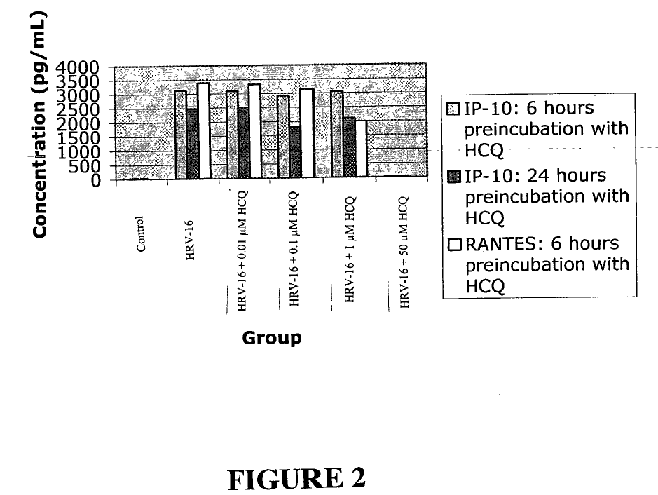

Rhinovirus capable of viral replication causes the release of the pro-inflammatory CXC chemokines, IP-10 and RANTES from infected epithelial cells. UV-inactivated rhinovirus, which cannot replicate does not result in release of CXC chemokines. The anti-malarial agents, including chloroquine and hydroxychloroquine block rhinovirus infection of epithelial cells. As seen in Table 1 and FIG. 1, primary epithelial cells treated with hydroxychloroquine and then infected with RV do not release the pro-inflammatory CXC chemokines which are a marker of active viral infection. Pre-incubation of immortalized epithelial cells (BEAS-2B) with anti-malarial compounds, such as hydroxychloroquine has the same effect as seen in Table 2 and FIG. 2.

Treatment with the anti-malarial compounds inhibits and prevents the elaboration of pro-inflammatory chemokines. For example, treatment with hydroxychloroquine blocks the elaboration of the pro-inflammatory chemokines, IP-10 and RANTES from both primary tonsillar and immortalized epithelial cell lines (Table 1 & 2, FIG. 1 & 2). Since these chemokines figure prominently in generation of inflammatory responses and symptoms, this effect has important therapeutic implications.

TABLE 1
Effect of HCQ on IP-10/RANTES Production in
Primary Human Epithelial Cells in pg/ml
with exposure to HRV-16

| | IP-10: | | | RANTES- | |
|---|---|---|---|---|---|
| | hours of | | | hours of | |
| | preincubation | | | preincubation | |
| Experiment | 24 | 48 | Experiment | 24 | 48 |
| Control | 31 | 31 | Control | 86 | 104 |
| HRV-16 | 1075 | 1400 | HRV-16 | 425 | 854 |
| HRV-16 + 50 | 31 | 112 | HRV-16 + HCQ | ND | 509 |
| microM HCQ | | | | | |

TABLE 2
Effect of varying concentrations of HCQ preincubation
on BEAS-2B epithelial cells in pg/ml exposed to
HRV-16 and assayed for IP-10 and RANTES

| | | | RANTES: |
|---|---|---|---|
| | IP-10: 6 hours | IP-10: 24 hours | 6 hours |
| | preincubation | preincubation | preincubation |
| Control | 31 | 31 | 0 |
| HRV-16 | 3123 | 2478 | 3388 |
| HRV-16 + 0.01 μM | 3084 | 2506 | 3326 |
| HCQ | | | |
| HRV-16 + 0.1 μM | 2914 | 1814 | 3128 |
| HCQ | | | |
| HRV-16 + 1 μM | 3045 | 2098 | 1994 |
| HCQ | | | |
| HRV-16 + 50 μM | 31 | 31 | 0 |
| HCQ | | | |

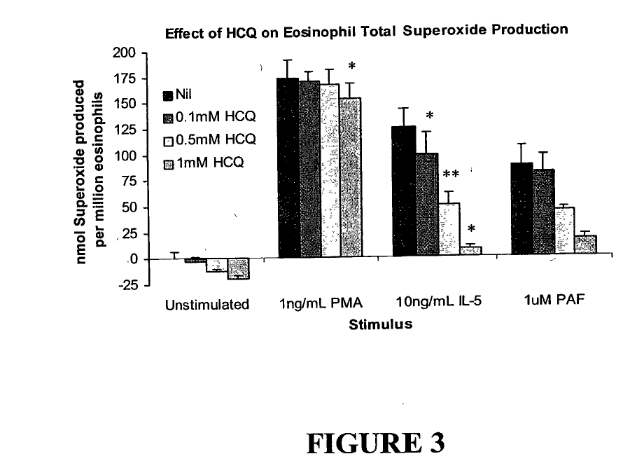

Anti-malarial agents also block the secondary aspects of rhinovirus infections. Without wishing to be bound, it is believed that they are inhibit by blocking the secondary events of viral infections. Release of lysosomal products, such as superoxides are potently inhibited by treatment with anti-malarials especially at concentrations between 0.1 mm and 100 mm. As seen in FIG. 3, IL-5 or PAF induced eosinophil superoxide is inhibited by HCQ but only at concentrations of at least 0.5 mM, or about 200 mcg/ml. Similar effects are seen on both treated neutrophils and mononuclear cells (NP Hurst Biochem Pharm 1986; 35:3083-89; NP Hurst Annals Rheum Dis 1987; 46:750-56) These effects are nearly immediate and require only 1 hour pre-incubation of lysosomal products such as superoxides which are also inhibited by anti-malarial agents.

It is noted that in their presently available oral forms, HCQ and other anti-malarial agents are universally considered slow acting drugs. In the treatment of rheumatic diseases, such as lupus erythematosus and rheumatoid arthritis, onset of action is characteristically 3-4 months. Charous presented convincing evidence (Charous, B L et al., J Allerg Clin Immunol, 1998; 102: 198-203) that therapeutic effect in asthma with oral HCQ begins only after 22 weeks of treatment. This delay in onset appears due to the requirement for active drug concentration in target organs before the onset of therapeutic effect. Hence, one requirement for drug action is time. The second requirement for onset of drug effect is that anti-malarial, e.g., HCQ achieve therapeutic concentration in the target organs. Inasmuch as HCQ has a notable selective distribution throughout body organs (McChesney, E W, “Animal toxicity and pharmacokinetics of hydroxchloroquine sulfate”, Amer J Med, 1983; July: 11-18), administration of HCQ or other anti-malarial per ora does not imply that the sufficient drug concentrations will reach the potential sites of epithelial infection in the nasopharynx and bronchial linings.

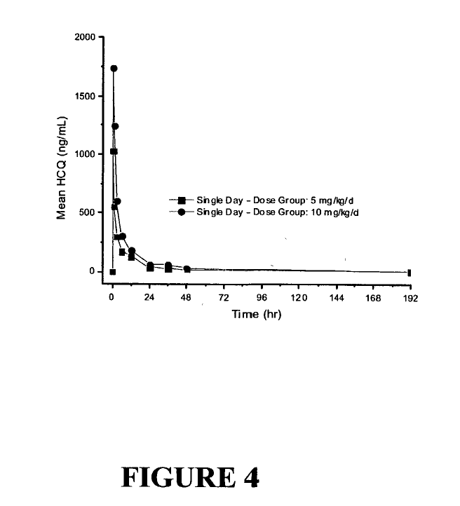

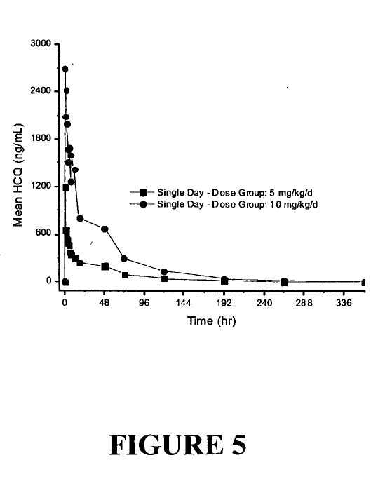

Accordingly, the present inventor has discovered that an anti-malarial agent administered in a local or targeted fashion, directly to the diseased organ or area of inflammation of a mammal, e.g., patient, is much more effective and efficacious than when administered in a conventional oral dosage with the result that the agent reaches a therapeutic level with surprising rapidity, in the targeted tissue organ, while the undesirable side effects are minimized. For purposes of illustration, the effects of targeted delivery as opposed to systemic delivery of a representative anti-malarial, hydroxychloroquine, for prevention of rhinovirus infection is compared. As shown in Tables 1 and 2 and in FIG. 3, the effective inhibitory concentrations of this agent ranges from about 50 to 1000 microM (or about 20 to 400 micrograms/ml). Safe oral dosing of anti-malarials, such as chloroquine and hydroxychloroquine yields serum levels far below these ranges: 0.6 to 0.9 microM for chloroquine and 1.4 to 1.5 microM for hydroxychloroquine (MacKenzie, A H, “Pharmacologic actions of the 4-aminoquinoline compounds”, Am J Med, 1983, July: 5-10). Moreover, the inventor has noted that oral or systemic administration of HCQ cannot provide adequate plasma levels of HCQ to achieveefficacious results. Even at doses nearly twice that used in humans, peak serum concentrations following intravenous of administration of 10 mg/kg HCQ in rats was only 2 mcg/ml (FIG. 4); in dogs, peak whole blood concentrations were less than 3 mcg/ml (FIG. 5).

In contrast, targeted treatment of the nasopharynx or bronchial airway with anti-malarials, such as HCQ can rapidly reach therapeutic concentrations. Experiments using aerosolized hydroxychloroquine in ascaris sensitized asthmatic sheep asthmatic demonstrated therapeutic effects with use of a total dose of only 10 mg/d about five percent of conventional oral dosing (See, Charous B L et al., “Aerosolized hydroxychloroquine (AHCQ) protects against antigen-induced early (EAR) and late airway responses (LAR) and airway hyperresponsiveness (AHR) in allergic sheep”, Am J Resp Crit Care Med 2001; 163: A859). The rapid onset of action of aerolized HCQ was not matched in animals treated with oral gavage, supporting the perceived advantages of targeted delivery of this compound.

Compounds suitable for the present invention are anti-malarial compounds. By anti-malarial, as used herein, it is meant that the drug has been historically belonged to the class of drugs known as anti-malarials. Preferred anti-malarials include quinolines, especially aminoquinolines, and more especially 8- and 4-aminoquinolines, acridines, e.g., 9-amino acridines and quinoline methanols, e.g., 4-quinolinemethanols. They also preferably have immunomodulatory and anti-inflammatory effects. Anti-malarial agents are well known in the art. Examples of anti-malarial agents can be found, for example, in GOODMAN AND GILMAN'S: THE PHARMACOLOGICAL BASIS OF THERAPEUTICS, chapters 45-47, pages 1029-65 (MacMillan Publishing Co. 1985), hereby incorporated by reference. The anti-malarial compound are preferably those which also exhibit an anti-inflammatory effect.

The preferred anti-malarial compounds are quinine based or are aminoquinolines, especially 4- and 8-amino quinolines. An especially preferred class of anti-malarials has a core quinoline structure (examples are mefloquine and quinine) which is usually substituted at one or more positions, typically at least at the 4- and/or 8-positions. One skilled in the art would understand that such agents could be administered in derivatized forms, such as pharmaceutically acceptable salts, or in a form that improves their pharmacodynamic profiles, such as esterification of acid or alcohol substituents with lower alkyls (e.g., C₁₋₆) or lower alkanoyloxy

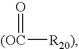

respectively, wherein R₂₀ is lower alkyl. Another class of anti-malarials, exemplified by quinacrine, is based on an acridine ring structure, and may be substituted in the manner described above.

Especially preferred compounds for use in the present invention are aminoquinolines, including 4-amino and 8-aminoquinolines and their derivatives (collectively, “aminoquinoline derivatives”) and aminoacridines, especially 9-amino acridines, which are described by the following formula:

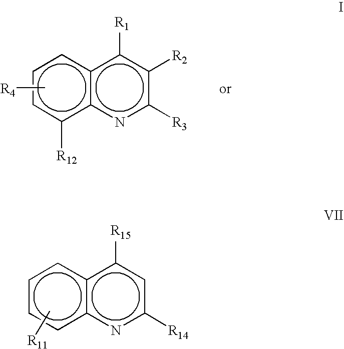

or pharmaceutically acceptable salts thereof, wherein

R₂ and R₃ are independently hydrogen, or lower alkyl or R₂ and R₃ taken together with the carbon atoms to which they are attached form an aryl ring, which ring may be unsubstituted or substituted with an electron withdrawing group or an electron donating group,

one of R₁ and R₁₂ is NHR₁₃ while the other is hydrogen;

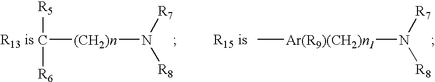

R₄, R₁₀, R₁₁ and R₁₄ are independently hydrogen or an electron donating group or electron withdrawing group;

R₅ and R₆, are independently hydrogen or lower alkyl which may be unsubstituted or substituted with an electron withdrawing or electron donating group;

R₇ and R₈ are independently hydrogen or lower alkyl, which may be unsubstituted or substituted with an electron withdrawing or electron donating group;

Ar is aryl having 6-18 ring carbon atoms;

R₉ is hydrogen or hydroxy or lower alkoxy or

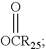

R₂₅ is lower alkyl or hydrogen; and

n and n₁ are independently 1-6.

As used herein, the terms “electron donating groups” and “electron withdrawing groups” refer to the ability of a substituent to donate or withdraw an electron, respectively, relative to that of hydrogen if the hydrogen atom occupied the same position in the molecule. These terms are well understood by one skilled in the art and are discussed in Advanced Organic Chemistry, by J. March, John Wiley & Sons, New York, N.Y., pp. 16-18 (1985) and the discussion therein is incorporated herein by reference. Electron withdrawing groups include halo, including bromo, fluoro, chloro, iodo and the like; nitro; carboxy; carbalkoxy; lower alkenyl; lower alkynyl; formyl; carboamido; aryl; quaternary ammonium compounds, and the like. Electron donating groups include such groups as hydroxy; lower alkoxy; including methoxy; ethoxy and the like; lower alkyl, such as methyl; ethyl, and the like; amino; lower alkylamino; diloweralkylamino; aryloxy, such as phenoxy and the like; arylalkoxy, such as benzyl and the like; mercapto, alkylthio, and the like. One skilled in the art will appreciate that the aforesaid substituents may have electron donating or electron withdrawing properties under different chemical conditions.

The term alkyl, when used alone or in conjunction with other groups, refers to an alkyl group containing one to six carbon atoms. It may be straight-chained or branched. Examples include methyl, ethyl, propyl, isopropyl, butyl, sec-butyl, isobutyl, tert-butyl, pentyl, neopentyl, hexyl and the like.

Lower alkoxy refers to an alkyl group which is attached to the main chain by an oxygen bridging atom. Examples include methoxy, ethoxy, and the like.

Lower alkenyl is an alkenyl group containing from 2 to 6 carbon atoms and at least one double bond. These groups may be straight chained or branched and may be in the Z or E form. Such groups include vinyl, propenyl, 1-butenyl, isobutenyl, 2-butenyl, 1-pentenyl, (Z)-2-pentenyl, (E)-2-pentyl, (Z)-4-methyl-2-pentenyl, (E)-4methyl-2-pentenyl allyl, pentadienyl, e.g., 1,3 or 2,4-pentadienyl, and the like. It is preferred that the alkenyl group contains at most two carbon-carbon double bonds, and most preferably one carbon-carbon double bond.

The term alkynyl include alkynyls containing 2 to 6 carbon atoms. They may be straight chain as well as branched. It includes such groups as ethynyl, propynyl, 1-butynyl, 2-butynyl, 1-pentynyl, 2-pentynyl, 3-methyl-1-pentynyl, 3-pentynyl, 1 hexynyl, 2-hexynyl, 3-hexynyl, and the like.

The term aryl refers to an aromatic group containing only carbon ring atoms which contains up to 18 ring carbon atoms and up to a total of 25 carbon atoms and includes the polynuclear aromatic rings. These aryl groups may be monocyclic, bicyclic, tricyclic, or polycyclic, and contain fused rings. The group includes phenyl, naphthyl, anthracenyl, phenanthranyl, xylyl, tolyl and the like.

The aryl lower alkyl groups include, for example, benzyl, phenethyl, phenpropyl, phenisopropyl, phenbutyl, diphenylmethyl, 1,1-diphenylethyl, 1,2diphenylethyl and the like.

The term halo include fluoro, chloro, bromo, iodo and the like.

The preferred values of R₂ and R₃ are independently hydrogen or alkyl containing 1-3 carbon atoms. It is most preferred that R₃ is hydrogen. It is most preferred that R₂ is hydrogen or alkyl containing 1-3 carbon atoms, especially methyl or ethyl. It is most preferred that R₂ is hydrogen or alkyl containing 1-3 carbon atoms or hydrogen and R₃ is hydrogen.

Alternatively, if R₂ and R₃ are taken together with the carbon atoms to which they are attached, it is most preferred that they form a phenyl ring. The phenyl ring is preferably unsubstituted or substituted with lower alkoxy, hydroxy, lower alkyl or halo.

It is preferred that R₄ is an electron withdrawing group, more specifically, halo, especially chloro, or is hydroxy or lower alkoxy. It is even more preferred that when R₁ is NHR₁₃, R₄ is substituted on the 7-position of the quinoline ring. It is most preferred that when R₁ is NHR₃, R₄ is halo.

However, when R₁₂ is NHR₃, it is preferred that R₄ is an electron donating group, such as hydroxy or alkoxy. More specifically, it is preferred that R₄ is methoxy or ethoxy when R₁₂ is NHR₁₃. It is even more preferred that R₄ is on the 6position of the quinoline ring when R₁₂ is NHR₃.

It is preferred that one of R₅ and R₆ is hydrogen and the other is lower alkyl. It is even more preferred that R₅ is hydrogen and R₆ is lower alkyl, especially alkyl containing 1-3 carbon atoms and most preferably methyl.

The preferred value of R₇ is lower alkyl, especially alkyl containing 1-3 carbon atoms and most preferably methyl and ethyl.

Preferred values of R₈ include lower alkyl containing 1-3 carbon atoms, and most preferably methyl and ethyl. However, it is preferred that the alkyl group is unsubstituted or if substituted, is substituted on the omega (last) carbon in the alkyl substituent. The preferred substituent is lower alkoxy and especially hydroxy.

The preferred R₉ is lower alkoxy and especially hydroxy.

R₁₁ is preferably an electron withdrawing group, especially trifluoromethyl. It is preferably located on the 8-position of the quinoline ring.

R₁₄ is preferably an electron withdrawing group, and more preferably trifluoromethyl. It is preferably present on the 2-position of the quinoline ring.

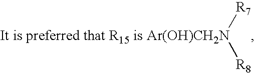

wherein R₇ and R₈ are independently alkyl containing 1-3 carbon atoms and Ar is phenyl.

In both R₁₃ and R₁₅, it is preferred that R₇ and R₈ contain the same number of carbon atoms, although one may be unsubstituted while the other is substituted. It is also preferred that R₇ and R₈ are the same.

The preferred value of n is 3 or 4 while the preferred value of n₁ is 1.

Preferred anti-malarials have the structure:

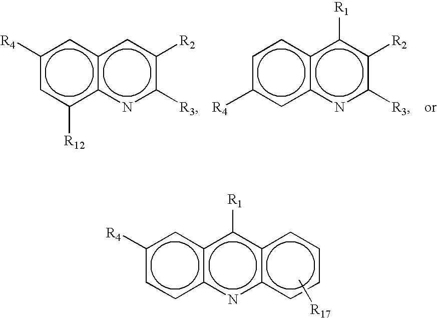

wherein R₁₂, R₄, R₂, R₃ and R₁ are as defined hereinabove and R₁₇ is hydrogen, halo, lower alkyl, lower alkoxy.

Preferred antimalarials include the 8-aminoquinolines, 9-aminocridines and the 7-chloro-4-aminoquinolines. Examples include pamaquine, primaquine, pentaquine, isopentaquine, quinacrine salts, 7-chloro-4-aminoquinolines, such as the chloroquines, hydroxychloroquines, sontoquine, amodiaquine and the like.

Another class of preferred antimalarial are cinchono alkaloids and 4quinoline methanols, such as those having the formula:

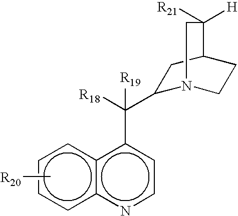

wherein one of R₁₈ and R₁₉ is hydroxy or loweralkylcarbonyloxy or hydrogen, and the other is H, and R₂₀ is hydrogen or loweralkoxy and R₂₁ is hydrogen or CH═CH₂.

Examples include rubane, quinine, quinidine, cinchoidine, epiquinine, epiquinidine, cinchonine, and the like.

Another preferred quinoline is a quinoline methanol, such as mefloquine or derivative thereof of the formula:

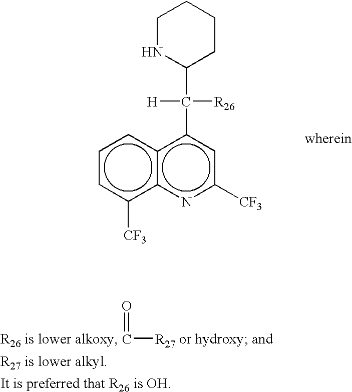

The most preferred anti-malarials include mefloquinine, and chloroquine and its congeners, such as hydroxychloroquine (HCQ), amodiaquine, pamaquine and pentaquine and pharmaceutically acceptable salts thereof.

The most preferred anti-malarial agent for the invention is hydroxychloroquine, shown below, or a pharmaceutically suitable salt thereof, such as hydroxychloroquine sulfate

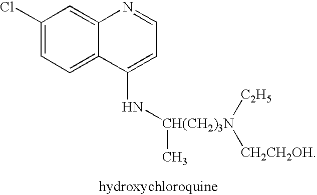

The antimalarials are commercially available or are prepared by art recognized techniques known in the art.

For example, the 4-aminoquinolines can be prepared as follows:

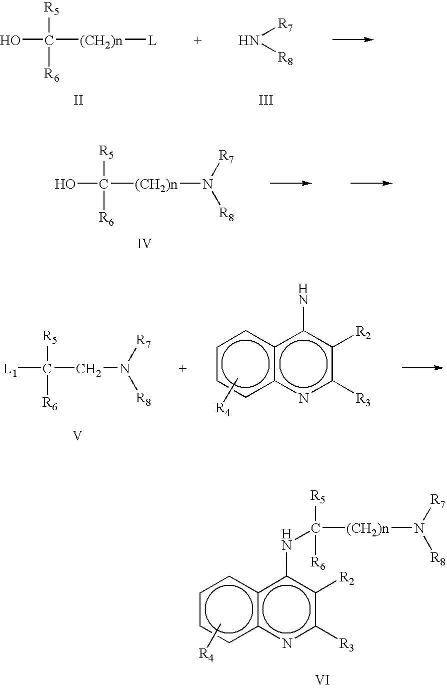

In the above scheme, R₁, R₂, R₃, R₄, R₅, R₆, R₇, R₈, and n are as defined hereinabove, and L and L₁ are good leaving groups, such as halides or sulfonates, e.g., mesylates or aryl sulfonates, e.g., tosylates, brosylates, and the like.

The compound of Formula II containing a leaving group, L, is reacted with the amine of Formula III under amine alkylation conditions. The alcohol group in the product of Formula IV (OH group) is converted to a leaving group by reactions known in the art. For example, sulfonic esters, such as tosylates, mesylates or brosylates are prepared by treatment of sulfonic halides of the formula R₂₃SO₂X₁ wherein X₁ is halide and R₂₃ is lower alkyl, such as methyl, aryl or substituted aryl, such as p-bromophenyl, p-tolyl with the alcohol of Compound IV. The reaction is usually effected in the presence of a weak base, such as pyridine. Alternatively, the alcohol can be converted to the corresponding halide by reaction of the alcohol of IV with HCl, HBr, thienyl chloride, PCl₃, PCl₅ or POCl₁₃. The product of V is then reacted under amine alkylation conditions with the quinoline amine to provide the 4-amino quinoline product.

The 9-aminoacridines and the 8-aminoquinoline are prepared similarly. More specifically, product V is reacted with

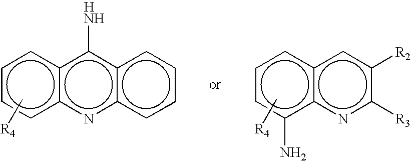

under amine alkylation reaction conditions.

The reactions described hereinabove are preferably conducted in solvents which are inert to the reactants and products and in which the reactants, are soluble, such as tetrahydrofuran, ethers, acetones, and the like. It is preferred that the solvents are volatile. The reactions are conducted at effective reaction conditions and are conducted at temperatures ranging from room temperature up to and including the reflux temperatures of the solvent.

An exemplary procedure for the preparation of compounds of Formula VII is as follows:

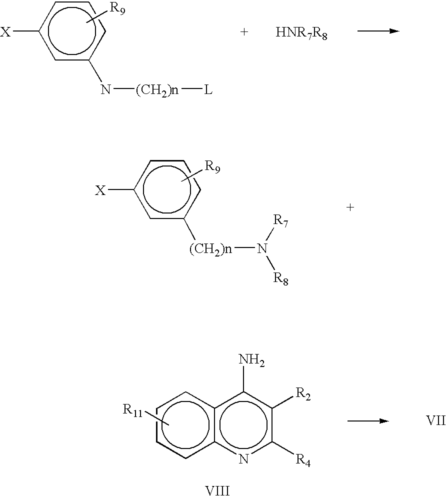

The first reaction is a simple amino alkylation reaction as described hereinabove. The product thereof is reacted with the amine of Formula VIII in the presence of a strong base such as amide to form the product of Formula VII.

Many of the compounds described hereinabove, especially the 4-quinoline methanols, can be converted to ethers by reacting the salt of the alcohols with an alkyl halide or arylalkyl halide or aryl halide to form the corresponding ether. Moreover, the esters can be formed from the hydroxy group by reacting the alcohol, such as the 4-quinoline methanol, with an alkanoic acid, arylalkonic acid or aryloic acid or acylating derivatives thereof in the presence of acid, for example, HCl, H₂SO₄ or p-toluene sulfonic acid under esterification conditions.

If any of the groups on R₁, R₂, R₃, R₄, R₅, R₆, R₇, R₈ are reactive with any of the reagents used or with any of the reactants or products, then they would be protected by protecting groups known in the art to avoid unwanted side reactions. This protecting groups normally used in synthetic organic chemistry are well known in the art. Examples are found in PROTECTIVE GROUPS IN ORGANIC SYNTHESIS, by T. W. Greene, John Wiley & Sons, Inc., NY 1981 (“Greene”), the contents of which are incorporated by reference.

As described hereinabove, the anti-malarial compounds used in the present invention are useful for the prophylaxis and treatment of diseases or maladies caused or associated with the infection of mammals by viruses, especially either adenoviruses or rhinoviruses. To prevent or treat those diseases, the anti-malarials are administered to the mammal in prophylatically or therapeutically effective amounts, respectively.

As used herein, the term “mammal” refers to a warm blooded vertebrate that belongs to the class Mammalia whose females have mammary glands to nourish their young. Examples include cats, dogs, horses, cows, pigs, mice, rats, and primates, including humans, and the like. The preferred mammal is humans.

The terms subject or patient, when used herein, are used interchangeably; both refer to mammals. The preferred subject or patient is human.

The term “prophylaxis” refers to the prevention or a measurable reduction in the likelihood of a patient acquiring a disease caused by or associated with viral infections, such as by human corona virus, influenza virus, adenovirus and/or rhinovirus, even if the animal is suffering from another malady or disease, such as asthma, which debilitates the animal and makes it more susceptible to adenovirus and/or rhinovirus infections. If a patient or mammal is suffering from a disease caused by or associated with viral infections, such as by human corona virus, influenza virus adenovirus and/or rhinovirus, the term also refers to the prevention of the disease from becoming exacerbated. When used in relation to a virus infection, it refers to the prevention or reduction in the likelihood of a mammal being infected by viruses, such as by human corona virus, influenza virus, adenovirus or rhinovirus, and the like.

The terms “treating”, “treat”, or “treatment”, as used herein, refers to the reduction and/or alleviation of at least one adverse effect or symptoms of a disease associated with caused by the virus, e.g., human corona virus, influenza virus, adenovirus or rhinovirus. It refers to the management and care of a mammalian subject, preferably human, for the purpose of combating the disease, condition or disorder and includes the administration of the anti-malarial compounds described herein to delay the onset of at least one symptom or complication associated with the disease, alleviating the symptom or effect or complications associated therewith or in the alternative eliminating the disease or condition. When used in relation to a virus infection, it refers to the reduction in or elimination of a viral infection, such as adenovirus or rhinovirus.

As used herein, “administering” refers to any method, which, in sound medical practice, delivers the compounds or compounds used.

The anti-malarials described herein are administered to mammals, e.g., humans for the prophylaxis or treatment of viral infections, such as adenovirus, rhinovirus, human corona virus, or influenza virus infections or any diseases or malady caused by associated with such infections. When treating these aforementioned infections or disease, they are administered to the mammal suffering therefrom with a therapeutically effective amount of the anti-malarial described herein. When used for the prophylaxis of the aforementioned infections or diseases, they are administered to the mammal in a prophylatically effective amount.

The terms “therapeutically effective amount” and “therapeutically effective amount” are used interchangeably and refer to an amount effective in eliminating or alleviating the infection by a virus such as an adenovirus, rhinovirus, influenza virus or human corona virus, and the like when referring to the treatment of an infection. These terms also refer to an amount effective in curing or alleviating the symptoms of a disease or malady caused by or associated with a viral, such as rhinovirus and/or adenovirus, human corona virus, influenza virus infection, when referring to a disease or malady.

The term “prophylatically effective amount” refers to an amount effective in preventing or reducing the likelihood of a mammal, e.g., human from being infected by a virus, e.g., adenovirus or rhinovirus when referring to the treatment of an infection. These terms also refer to the amount effective in preventing or reducing the likelihood of a mammal, e.g., human acquiring a disease or malady caused by or associated with viral, such as adenovirus or rhinovirus, human corona virus or influenza virusinfection, when referring to a disease or malady. In addition, in the latter context, it also refers to the amount effective in preventing a mammal afflicted with a disease or malady caused by or associated with a viral infection, such as adenovirus or rhinovirus infection from worsening or becoming more severe or in making the disease.

The physician will determine the dosage of the antimalarial compounds which will be most suitable for the mammal, e.g., patient, and it will vary with the form of administration and the particular compound chosen. Furthermore, it will vary depending upon various factors, including but not limited to the patient under treatment, the age of the patient, the severity of the condition being treated and the like. He will generally wish to initiate treatment with small dosages substantially less than the optimum dose of the compound and increase the dosage by small increments until the optimum effect under the circumstances is reached.

For the prophylaxis use, it is preferred that the anti-malarial compounds described herein are administered in an effective airway delivered dose of about 2 to about 10 mg per day and more preferably about 2 to about 5 mg per day. For therapeutic use, it is preferred that the anti-malarial compounds described herein be administered in an effective airway delivery of about 2 to about 10 mg per day and more preferably about 2 to about 5 mg per day. The desired dose can be taken all at once, or it may be administered two, three, four, five, six or more, sub-doses administered at appropriate intervals throughout the day.

The anti-malarials compounds described herein are formulated for localized (targeted) delivery for administration thereof to internal organs, such as the lungs, or the eye, or internal muscles or tissues, by local or targeted delivery. “Local or topical delivery” and “locally administering” are used in this description to denote direct delivery to the site, such that the anti-malarials act directly on affected tissue or the area of a diseased organ. Local delivery contrasts with methods by which a drug is administered orally, or otherwise systemically, and is absorbed into the circulation for distribution throughout the patient's body. Examples of local delivery include inhalation, nasal spray, and eye drops and by injections directly to the organ, muscle or tissue. It is to be noted that local delivery excludes intravenous injection, e.g., injected intravenously, into the circulatory blood of the patient. Topical delivery to the skin, moreover, is not contemplated in the practice of “local or topical delivery” as defined above. These compositions may be solutions, suspensions and admixtures, for example. As one having ordinary skill in the art would understand, they may be prepared essentially as detailed in REMINGTON'S PHARMACEUTICAL SCIENCES, 18^{th} ed., (Mack Publishing Co. 1990) (“Remingtons”), which is hereby incorporated by reference.

For pulmonary delivery, a therapeutic composition of the invention is formulated and administered to the patient in solid or liquid particulate form by direct administration e.g., inhalation into the respiratory system.

Solid or liquid particulate forms of the active compound prepared for practicing the present invention include particles of respirable size: that is, particles of a size sufficiently small to pass through the mouth and larynx upon inhalation and into the bronchi and alveoli of the lungs. In general, particles ranging from about 1 to 10 microns in size are within the respirable range. The pharmaceutical compositions containing the anti-malarial compounds are preferably administered by direct inhalation into the respiratory system for delivery as a mist or other aerosol or dry powder. Particles of non-respirable size which are included in the aerosol tend to be deposited in the throat and swallowed; thus the quantity of non-respirable particles in the aerosol is preferably minimized.

The dosage of the anti-malarials via this route will vary depending on the condition being treated and the state of the subject, but generally may be an amount sufficient to achieve dissolved concentrations of anti-malarial compound on the airway surfaces of the subject. Depending upon the solubility of the particular formulation of anti-malarial administered, the daily dose may be divided among one or several unit dose administrations. The daily dose by weight will depend upon the age and condition of the subject. Such a daily dose of the anti-malarial compound ranges from about 2-10 mg delivered to the site in an effective airway delivery system and more preferably from about 2 to about 5 mg delivered to the site in an effective airway delivery system. In the most preferred embodiments, only one dose is administered to the patient per day. The doses of the anti-malarial compounds may be provided as one or several prepackaged units.

In the manufacture of the preferred local formulation, in accordance with the description herein, the anti-malarial compounds or the pharmaceutically acceptable salts are typically admixed with, among other things, an acceptable carrier. The carrier must, of course, be acceptable in the sense of being compatible with any other ingredients in the formulation and must not be deleterious to the patient. The carrier may be a solid or a liquid, or both, and is preferably formulated with the compound as a unit-dose formulation. One or more drugs may be incorporated in the formulations of the invention, which formulations may be prepared by any of the well-known techniques of pharmacy consisting essentially of admixing the drug with the other various components described hereinbelow present therein.

Aerosols of liquid particles comprising the anti-malarial compounds may be produced by any suitable means, such as inhalatory delivery systems. One is a traditional nebulizer which works in a mechanism similar to the familiar perfume atomizer. The airborne particles are generated by a jet of air from either a compressor or compressed gas cylinder-passing through the device (pressure driven aerosol nebulizer). In addition, newer forms utilize an ultrasonic nebulizer by vibrating the liquid at speed of up to about 1 MHz. See, e.g., U.S. Pat. No. 4,501,729, the contents of which are incorporated by reference. Nebulizers are commercially available devices which transform solutions or suspensions of the anti-malarial into a pharmaceutical aerosol mist either by means of acceleration of compressed gas, typically air or oxygen, through a narrow venturi orifice or by means of ultrasonic agitation. Suitable formulations for use in nebulizers consist of the anti-malarials in a liquid carrier. The carrier is typically water (and most preferably sterile, pyrogen-free water) or a dilute aqueous alcoholic solution, preferably made isotonic but may be hypertonic with body fluids by the addition of, for example, sodium chloride. Optional additives include preservatives if the formulation is not made sterile, for example, methyl hydroxybenzoate, as well as antioxidants, flavoring agents, volatile oils, buffering agents and surfactants, which are normally used in the preparation of pharmaceutical compositions. For nebulizer use it is preferred than the anti-malarial compounds, e.g., HCQ are dissolved in sterile water with the pH adjusted to 7.4-7.6 and sodium chloride is added to achieve isotonic conditions.

Aerosols of solid particles comprising the anti-malarial compound may likewise be produced with any solid particulate medicament aerosol generator. Aerosol generators for administering solid particulate medicaments to a subject produce particles which are respirable, as explained above, and generate a volume of aerosol containing a predetermined metered dose of a medicament at a rate suitable for human administration. One illustrative type of solid particulate aerosol generator is an insufflator. Suitable formulations for administration by insufflation include finely comminuted powders which may be delivered by means of an insufflator or taken into the nasal cavity in the manner of a snuff. In the insufflator, the powder (e.g., a metered dose thereof effective to carry out the treatments described herein) is contained in capsules or cartridges, typically made of gelatin or plastic, which are either pierced or opened in situ and the powder delivered by air drawn through the device upon inhalation or by means of a manually-operated pump. The powder employed in the insufflator consists either solely of the anti-malarial compound or of a powder blend comprising the anti-malarial compound, a suitable powder diluent, such as lactose, and an optional surfactant. A second type of illustrative aerosol generator comprises a metered dose inhaler. Metered dose inhalers are pressurized aerosol dispensers, typically containing a suspension or solution formulation of the anti-malarial compound in a liquified propellant. During use, these devices discharge the formulation through a valve, adapted to deliver a metered volume, from 10 to 22 microliters to produce a fine particle spray containing the anti-malarial compound. Suitable propellants include certain chlorofluorocarbon compounds, for example, dichlorodifluoromethane, trichlorofluoromethane, dichlorotetrafluoroethane and mixtures thereof. The formulation may additionally contain one or more co-solvents, for example, ethanol, surfactants, such as oleic acid or sorbitan trioleate, antioxidants and suitable flavoring agents.

Any propellant may be used in carrying out the present invention, including both chlorofluorocarbon-containing propellants and non-chlorofluorocarbon-containing propellants. Fluorocarbon aerosol propellants that may be employed in carrying out the present invention including fluorocarbon propellants in which all hydrogen are replaced with fluorine, chlorofluorocarbon propellants in which all hydrogens are replaced with chlorine and at least one fluorine, hydrogen-containing fluorocarbon propellants, and hydrogen-containing chlorofluorocarbon propellants. Examples of such propellants include, but are not limited to: CF₃CHFCF₂, CF₃CH₂CF₂H, CF₃CHFCF₃, CF₃CH₂CF₃, CF₃CHCl—CF₂Cl, CF₃CHCl—CF₃ CF₃CHF—CF₂Cl, and the like. A stabilizer such as a fluoropolymer may optionally be included in formulations of fluorocarbon propellants, such as described in U.S. Pat. No. 5,376,359 to Johnson.

Compositions containing respirable dry particles of micronized anti-malarial compounds may be prepared by grinding the dry active compound, with e.g., a mortar and pestle or other appropriate grinding device, and then passing the micronized composition through a 400 mesh screen to break up or separate out large agglomerates.

The aerosol, whether formed from solid or liquid particles, may be produced by the aerosol generator at a rate of from about 10 to 150 liters per minute. Aerosols containing greater amounts of medicament may be administered more rapidly. Typically, each aerosol may be delivered to the patient for a period from about 30 seconds to about 20 minutes, with a delivery period of about 1 to 5 minutes being preferred.

The particulate composition comprising the anti-malarial compound may optionally contain a carrier which serves to facilitate the formation of an aerosol. A suitable carrier is lactose, which may be blended with the active compound in any suitable ratio.

For example, hydroxychloroquine sulfate is a colorless crystalline solid which is readily soluble in water. Inhaled liquid forms may be formulated to contain such additives as are typically used in such pharmaceutical preparations, including, but not limited to an acceptable excipient and/or surfactant. A composition of the anti-malarial, e.g., HCQ, may be pre-formulated in liquid form, or prepared for the addition of a suitable carrier, like sterile water or physiological saline, immediately prior to use. The aerosol containing HCQ typically contain a propellant especially a fluorocarbon propellant. See Remington's, chapter 92. A particularly useful composition of HCQ is formulated in a nebulizer, for the treatment of a variety of pulmonary conditions. For the preparation of HCQ in inhaled powder form, the compound is finely divided, or micronized to enhance effectiveness, and admixed with a suitable filler. Inhaled powders may contain a bulking agent and/or stabilizer, as described hereinabove. Id., chapter 88. An insufflator (powder blower) may be employed to administer the fine powder.

The anti-malarial compounds may be administered by other methods of local delivery, as defined herein. Compositions for these other modes of local delivery may include sterile aqueous solutions which may also contain buffers, diluents and other suitable additives and may be administered in other forms, such as oral pastes or ointment, retention enemas, suppositories, and injectable solutions, which injectable solutions are administered directly to internal organs or tissues and not intravenously.

The anti-malarial compounds may, where appropriate, be conveniently present in discrete unit dosage forms and may be prepared by any of the methods well known in the art of pharmacy. Such methods include the step of bringing into association the active compound, i.e., the anti-malarial compound with liquid carriers, solid matrices, semi-solid carriers, finely divided solid carriers or combinations thereof, and then, if necessary, shaping the product into the desired delivery system. Methods for admixing a pharmaceutical with a carrier are known in the art and are applicable to the present formulation.

The anti-malarial compounds may also be formulated as an ophthalmic product. Such formulations for ophthalmic administration include eye drops and ophthalmic ointments, creams, suspensions and lotions. Drops, such as eye drops or nose drops, may be formulated with an aqueous or non-aqueous base also comprising one or more dispersing agents, solubilizing agents or suspending agents. Drops can be delivered via a simple eye dropper-capped bottle or eye-dropper, or via a plastic bottle adapted to deliver liquid contents dropwise, via a specially shaped closure. Ophthalmic preparations typically contain at least one anti-malarial compound in a sterile isotonic solution, for example, sodium chloride or boric acid. They may contain agents that increase viscosity, like methylcellulose, polyvinyl alcohol or hydroxymethyl cellulose.

For example, drops according to the present invention may comprise sterile aqueous or oily solutions or suspensions and may be prepared by dissolving the anti-malarial compound in a suitable aqueous solution of a bactericidal and/or any other suitable preservative. The resulting solution may then be clarified by filtration, transferred to a suitable container which is then sealed and sterilized by autoclaving. Alternatively, the solution may be sterilized by filtration and transferred to the container by an aseptic technique. Examples of bactericidal and fungicidal agents suitable for inclusion in the drops are phenylmercuric nitrate or acetate (0.002%), benzalkonium chloride (0.01%) and chlorhexidine acetate (0.01%).

Lotions include sterile aqueous solutions optionally containing a preservative and may be prepared by methods similar to those for the preparation of drops.

Creams or ointments are semi-solid formulations of the active ingredient particularly for ophthalmic application. They may be made by mixing the anti-malarials in finely-divided or powdered form alone or in solution or suspension in an aqueous or non-aqueous fluid, with a greasy or non-greasy basis. The basis may comprise hydrocarbons such as hard, soft or liquid paraffin, glycerol, beeswax, a metallic soap; a mucilage, an oil of natural origin such as almond, corn, arachis, castor or olive oil, wool fat or its derivatives, or a fatty acid such as stearic or oleic acid together with an alcohol such as propylene glycol or macrogols. The formulation may incorporate any suitable surface active agent such as sorbitan esters or polyoxyethylene derivative thereof. Suspending agents such as natural gums, cellulose derivatives or inorganic material such as silicaceous silicas; and other ingredients such as lanolin may also be included.

The anti-malarials compounds also may be formulated advantageously as nasal sprays, oral pastes, ointments to be administered directly to the organ, such as the eye, and retention enemas, and other means known to one of ordinary skill in the art for local delivery.

The pharmaceutical forms suitable for injectable use directly into muscle or tissue include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents, delaying absorption, for example, aluminum monostearate and gelatin.

Sterile injectable solutions are prepared by incorporating the anti-malarial compound in the required amount in the appropriate solvent with various of the other ingredients enumerated above, as required followed by filtered sterilization. Generally, dispersions are prepared by incorporating the sterilized anti-malarial compound into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the anti-malarial compound plus any additional desired ingredient from previously sterile-filtered solution thereof.

As used herein, “pharmaceutically acceptable carrier” includes any and all solvents, dispersion media, coatings, antibacterial and antifungal agents, isotonic and absorption delaying agents, and the like. The use of such media and agents for pharmaceutical active substances is well known in the art. Except insofar as any conventional media or agent is incompatible with the active ingredient, its use in the therapeutic compositions is contemplated. More than one anti-malarial compound can also be incorporated into the pharmaceutical compositions.

It is especially advantageous to formulate local compositions in dosage unit form for ease of administration and uniformity of dosage. Dosage unit form as used herein refers to physically discrete units suited as unitary dosages for the mammalian subjects; each unit containing a predetermined quantity of anti-malarial compound calculated to produce the desired therapeutic or prophylatic effect in association with the required pharmaceutical carrier.

The anti-malarials of the present application are useful for the treatment and prophylaxis of viral infections, including adenoviruses, rhinoviruses, human corona virus and/or influenza virus when administered in effective amounts. In addition, they are also useful for the treatment and prophylaxis of diseases caused by or associated with infections by influenza virus, human corona virus, adenoviruses and/or rhinoviruses. Many of these diseases were described hereinabove. For example, the anti-malarials are useful for the treatment and prophylaxis of colds. They are also useful for reducing the incidence of complications of primarily human corona virus, influenza virus, adenoviral and rhinoviral infections, such as bronchitis, sinusitis and otitis media. The anti-malarials described herein are also useful for reducing the incidence of complications of other diseases which may be exacerbated by the primary adenoviral, rhinoviral, human corona virus or influenza virus infections, such as those associated with pharyngitis, coughing and conjunctivitis, pharyngoconjunctival fever, and pertussis syndrome, haemorrhagic cystitis, meningitis, diarrhea, and the like.

The above preferred embodiments are given to illustrate the scope and spirit of the present invention. The embodiments described herein will make apparent to those skilled in the art other embodiments. These other embodiments are within the contemplation of the present invention. Therefore, the present invention should be limited only by the appended claims.

What is claimed is:

1. A method for the prophylaxis of an infection in a mammal of a virus said method comprising administering for targeted delivery to said mammal a prophylatically effective amount of an anti-malarial compound which is aminoquinoline or hydroxyquinoline.

2. The method according to claim 1 wherein the virus is adenovirus, rhinovirus, human corona virus or influenza virus.

3. A method for the treatment of an infection in a mammal from a virus said method comprising administering for targeted delivery to a mammal infected with said virus a therapeutically effective amount of an anti-malarial compound which is aminoquinoline or hydroxyquinoline.

4. The method according to claim 3 wherein the virus is adenovirus, rhinovirus, human corona virus or influenza virus.

5. A method for the prophylaxis of a disease in a mammal caused by or associated with an infection by a virus said method comprising administering for targeted delivery to said mammal a prophylatically effective amount of an anti-malarial compound which is aminoquinoline or hydroxyquinoline.

6. A method for the treatment of a disease in a mammal caused by or associated with an infection by a virus, said method comprising administering for targeted delivery to a mammal suffering from said diseases a therapeutically effective amount of an anti-malarial compound which is aminoquinoline or hydroxyquinoline.

7. The method according to claim 5 wherein the virus is adenovirus, rhinovirus, human corona virus or influenza virus.

8. The method according to claim 6 wherein the virus is adenovirus, rhinovirus, human corona virus or influenza virus.

9. The method according to claim 1, 3, 5 or 6 wherein the anti-malarial compound is an aminoquinoline.

10. The method according to claim 9 wherein said aminoquinoline has the formula: 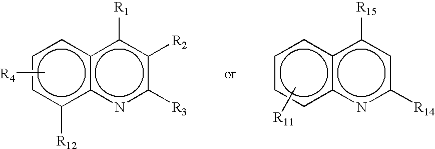
or pharmaceutically acceptable salts thereof, wherein
R₂ and R₃ are independently hydrogen, or lower alkyl or R₂ and R₃ taken together with the carbon atoms to which they are attached form an aryl ring, which aryl ring is unsubstituted or substituted with an electron withdrawing group or an electron donating group,
one of R₁ and R₁₂ is NHR₁₃ while the other is hydrogen; 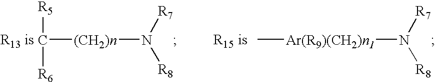
R₄, R₁₀, R₁₁ and R₁₄ are independently hydrogen or an electron donating group or electron withdrawing group;
R₅ and R₆, are independently hydrogen or lower alkyl which may be unsubstituted or substituted with an electron withdrawing or electron donating group;
R₇ and R₈ are independently hydrogen or lower alkyl, which may be unsubstituted or substituted with an electron withdrawing or electron donating group;
Ar is aryl having 6-18 ring carbon atoms which may be unsubstituted or substituted with an electron donating or electron withdrawing group;
R₉ is hydrogen or hydroxy or lower alkoxy or 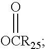
R₂₅ is lower alkyl or hydrogen; and
n and n₁ are independently 1-6.

11. The method according to claim 10 wherein the aminoquinoline is of the formula:

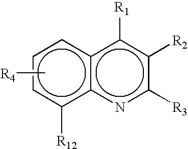

12. The method according to claim 11 wherein R₁ is NHR₁₃ and R₁₂ is hydrogen.

13. The method according to claim 12 wherein R₅ is hydrogen and R₆ is lower alkyl.

14. The method according to claim 12 wherein R₅ is hydrogen and R₆ is methyl.

15. The method according to claim 12 wherein n is 3.

16. The method according to claim 12 wherein R₃ is hydrogen.

17. The method according to claim 12 wherein R₄ is substituted in the 7-position of the quinoline ring.

18. The method according to claim 14 wherein R₄ is 7-halo.

19. The method according to claim 18 wherein halo is chloro.

20. The method according to claim 12 wherein R₇ is ethyl and R₈ is ethyl or 2-hydroxy ethyl.

21. The method according to claim 11 wherein R₁₂ is NHR₁₃ and R₁ is hydrogen.

22. The method according to claim 21 wherein R₅ is hydrogen and R₆ is lower alkyl.

23. The method according to claim 22 wherein R₅ is hydrogen and R₆ is methyl.

24. The method according to claim 21 wherein n is 3.

25. The method according to claim 22 wherein R₇ is hydrogen, methyl or ethyl and R₈ is hydrogen, methyl, ethyl, propyl or isopropyl.

26. The method according to claim 21 wherein R₄ is substituted on the 6-position of the quinoline ring.

27. The method according to claim 26 wherein R₄ is 6-lower alkoxy.

28. The method according to claim 27 wherein R₄ is 6-methoxy.

29. The method according to claim 10 wherein the amino quinoline has the formula: 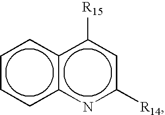
wherein R₁₅ is Ar (R₉)(CH₂)ₙ₁—N(R₇)(R₈).

30. The method according to claim 29 wherein Ar is phenyl.

31. The method according to claim 29 wherein R₉ is hydroxy.

32. The method according to claim 29 wherein R₁₅ is

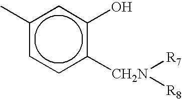

33. The method according to claim 29 wherein R₇ and R₈ are independently lower alkyl.

34. The method according to claim 33 wherein R₇ and R₈ are both ethyl

35. The method according to any one of claims 1, 3, 5 or 6 wherein the anti-malarial compound has the formula: 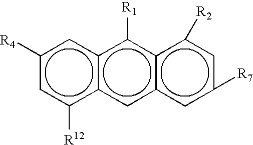
wherein
R₂ is hydrogen or lower alkyl;
one of R₁ and R₁₂ is NHR₁₃ while the other is hydrogen; 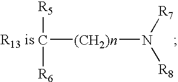
R₄ is hydrogen or an electron donating group or electron withdrawing group;
R₅ and R₆, are independently hydrogen or lower alkyl which may be unsubstituted or substituted with an electron withdrawing or electron donating group;
R₇ and R₈ are independently hydrogen or lower alkyl, which may be unsubstituted or substituted with an electron withdrawing or electron donating group; and
n is independently 1-6.

36. The method according to any one of claims 1-6 wherein the anti-malarial agent is pomaquine, primaquine, pentaquinine, isopentaquine, quinacrine salt, chloroquine, hydroxychloroquine, sontoquine, amodiaquine, mefloquine, or mepacrine or pharmaceutically acceptable salts thereof.

37. The method according to any one of claims 1-6 wherein the anti-malarial compound is hydroxychloroquine, chloroquine, mepacrine, mefloquinine, or pharmaceutically acceptable salts thereof.

38. The method according to any one of claims 1-6 wherein the anti-malarial compound is hydroxychloroquine or a pharmaceutically acceptable salt thereof.

39. The method according to claim 5 or 6 wherein the disease is a cold, bronchitis, sinusitis, or respiratory infection.

40. The method according to any one of claims 1-6 wherein the anti-malarial compound is administered by inhalation.

41. The method according to any one of claims 1-6 wherein the anti-malarial compound is administered in a nasal spray, eye drop, aerosol, ophthalmic ointment, cream, suspension or lotion.

42. The method according to any one of claims 1-6 wherein the anti-malarial compound is administered for targeted delivery in the respiratory epithelium.

43. A method of treating or preventing rhinoviral infection in a mammal comprising administering to said mammal an anti-viral effective amount of an anti-malarial compound.

44. The method according to claim 43 wherein the anti-malarial compound is aminoquinoline or hydroxyquinaline.

45. The method according to claim 44 wherein the aminoquinoline has the formula 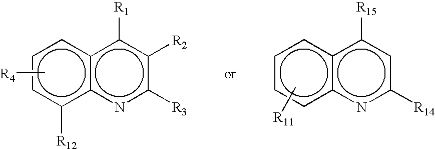
or pharmaceutically acceptable salts thereof, wherein
R₂ and R₃ are independently hydrogen, or lower alkyl or R₂ and R₃ taken together with the carbon atoms to which they are attached form an aryl ring, which aryl ring is unsubstituted or substituted with an electron withdrawing group or an electron donating group,
one of R₁ and R₁₂ is NHR₁₃ while the other is hydrogen; 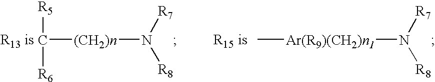
R₄, R₁₀, R₁₁ and R₁₄ are independently hydrogen or an electron donating group or electron withdrawing group;
R₅ and R₆, are independently hydrogen or lower alkyl which may be unsubstituted or substituted with an electron withdrawing or electron donating group;
R₇ and R₈ are independently hydrogen or lower alkyl, which may be unsubstituted or substituted with an electron withdrawing or electron donating group;
Ar is aryl having 6-18 ring carbon atoms which may be unsubstituted or substituted with an electron donating or electron withdrawing group;
R₉ is hydrogen or hydroxy or lower alkoxy or 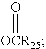
R₂₅ is lower alkyl or hydrogen; and
n and n₁ are independently 1-6.

46. The method according to claim 45 wherein the aminoquinoline is of the formula

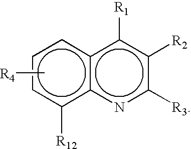

47. The method according to claim 46 wherein R₁ is NHR₁₃ and R₁₂ is hydrogen.

48. The method according to claim 46 wherein R₁₂ is a NHR₁₃ and R₁ is hydrogen.

49. The method according to claim 45 wherein R₁₅ is

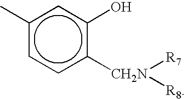

50. The method according to any one of claim 43 wherein the anti-malarial compound is hydroxychloroquine, chloroquine, mepacrine, mefloquinine, or pharmaceutically acceptable salts thereof.

51. The method according to any one of claims 43 wherein the anti-malarial compound is hydroxychloroquine or a pharmaceutically acceptable salt thereof.

52. A method for the treatment or prophylaxis of a disease in a mammal caused by or associated with an infection by a rhinovirus, comprising administering to said mammal a pharmaceutically effective amount of an anti-malarial compound.

53. The method according to claim 52 wherein the disease is a cold, bronchitis, sinusitis, or respiratory infection.

54. The method according to any one of claims 43 wherein the anti-malarial compound is administered by inhalation.

55. The method according to any one of claims 43 wherein the anti-malarial compound is administered in a nasal spray, eye drop, aerosol, ophthalmic ointment, cream, suspension or lotion.

56. The method according to any one of claims 43 wherein the anti-malarial compound is administered for targeted delivery in the respiratory epithelium.
